(19) United States
(12) Patent Application Publication
(10) Pub. No.: US 2021/0106671 A1
(43) Pub. Date: Apr. 15, 2021

(54) TREATMENT AND PREVENTION OF PLANTAR WARTS

(71) Applicant: Jack Silvers, Los Angeles, CA (US)

(72) Inventor: Jack Silvers, Los Angeles, CA (US)

(21) Appl. No.: 17/016,264

(22) Filed: Sep. 9, 2020

Related U.S. Application Data
(60) Provisional application No. 62/897,392, filed on Sep. 9, 2019.

(51) Int. Cl. A61K 39/12; A61K 33/00; A61K 31/34; A61K 31/60; A61K 31/19; A61K 31/343; A61K 31/215; A61K 31/513; A61K 38/14; A61B 18/02

(52) U.S. Cl. CPC A61K 39/12; A61K 33/00; A61K 31/34; A61K 31/60; A61K 31/19; A61B 2018/0047; A61K 31/215; A61K 31/513; A61K 38/14; A61B 18/0218; A61K 31/343

(57) ABSTRACT

Described herein are methods of treating or preventing plantar warts comprising administering to a subject a therapeutically effective amount of a vaccine against human papillomavirus (HPV) and reducing exposure of an area susceptible to plantar warts to moisture, to non-neutrally electrically charged materials, or a combination thereof. Also described are kits or articles of manufacture for use in treating or preventing plantar warts.

### FIELD OF THE INVENTION

Disclosed herein are methods of treating and preventing plantar warts.

### BACKGROUND OF THE INVENTION

The human papillomavirus (HPV) family consists of over 170 different DNA viruses, and most individuals harboring HPV exhibit no observable manifestation and may even resolve without the subject realizing that they harbored the virus. But some individuals harboring HPV can be afflicted with lesions or other growths, and in some cases, such as HPV-mediated cervical cancer, symptoms do not manifest for years after exposure to the virus. Often associated with sexually transmitted diseases, one individual can transmit HPV to a second individual by nonsexual transmission, such entry into the second individual through cuts, stratum corneum erosion, or epidermal abrasions. Plantar warts (also known as verruca plantaris) are a common manifestation of HPV subtypes 1, 2, 4, 57, 60, 63, 65, 66, and 156, with the warts typically appearing on the bottom of an affected individual's feet.

Plantar warts can resolve over time if left untreated, but affected individuals have to endure the physical discomfort as well as the unsightly appearance of the warts. For these reasons, individuals often seek medical treatment for their plantar warts, and currently available options such as cryotherapy, laser removal, topical medicines, and surgical removal can be expensive and time consuming. Other remedies, such as over-the-counter remedies, are often pursued by those affected with plantar warts as less costly and more convenient alternatives. Unfortunately, none of these treatments is also entirely effective, and recurrence of warts is an issue many patients experience while harboring HPV.

Thus, there is a need for improved methods for treating and preventing plantar warts. The disclosed methods are directed to these and other important needs.

### SUMMARY OF THE INVENTION

Disclosed herein are methods for preventing plantar warts on an area of a subject susceptible to plantar warts comprising administering to the subject a therapeutically effective amount of a vaccine against human papillomavirus (HPV); and reducing exposure of the area susceptible to plantar warts to moisture, non-neutrally electrically charged materials, or a combination thereof. Examples of non-neutrally electrically charged materials include, but are not limited to, rubber and plastic. In some embodiments, the moisture to which exposure is reduced is sweat moisture.

Also provided are methods of preventing the onset of plantar warts on an area of a subject susceptible to plantar warts comprising administering to the subject a therapeutically effective amount of a vaccine against human papillomavirus (HPV); and instructing the subject to reduce exposure of the area susceptible to plantar warts to sweat moisture.

Also disclosed herein are methods for preventing recurrence of plantar warts on an area of a subject previously affected with plantar warts comprising administering a drying agent or an absorbent composition to the area previously affected with plantar warts. In some embodiments, the absorbent composition will be neutrally electrically charged.

Methods are also provided for treating plantar warts on an affected area on a subject comprising removing at least one plantar wart from the affected area on the subject and administering a drying agent or absorbent composition to the affected area.

Disclosed herein are methods of preventing plantar warts on an area of a subject susceptible to plantar warts comprising reducing exposure of an area susceptible to plantar warts to moisture. In some aspects, the moisture is sweat.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

When a range of values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods, which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include the plural.

The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

As used herein, “affected area” refers to any area on the body where a plantar wart is or was present on an individual. In most cases, affected areas will be the hands or feet of an individual, but other areas of the body may also be affected. Affected areas will often include visible regions of the skin

The term “subject” as used herein refers to any animal, but in particular humans. Thus, the methods are applicable to human and nonhuman animals, although preferably used most preferably with humans. “Subject” and “patient” are used interchangeably herein.

As used herein, the phrase “therapeutically effective amount” refers to an amount of a composition (e.g., vaccine, pharmaceutical, or topical treatment) as described herein, effective to achieve a particular biological or therapeutic result such as, but not limited to, biological or therapeutic results disclosed, described, or exemplified herein. The therapeutically effective dose may vary according to factors such as the length of time an individual has harbored HPV; the age, sex, and/or weight of the individual; and the ability of the composition to cause a desired response in a subject. Such results include, but are not limited to, the treatment of plantar warts, as determined by any means suitable in the art.

As used herein, “treating” and like terms refer to clearing clinically detectable lesions resulting from HPV infection; reducing the severity and/or frequency of plantar warts; eliminating plantar warts; reducing the likelihood of plantar warts; and/or improving or remediating damage caused, directly or indirectly, by HPV-associated plantar warts.

Some embodiments of the present disclosure provide methods of preventing plantar warts on an area of a subject susceptible to plantar warts comprising administering to the subject a therapeutically effective amount of a vaccine against HPV and reducing exposure of the area susceptible to plantar warts to moisture, non-neutrally electrically charged materials, or a combination thereof. In some aspects, the reducing exposure to moisture comprises reducing exposure to sweat. For example, sweat moisture can be absorbed by some articles of clothing such as cotton socks. In some aspects, neutrally charged articles of clothing or other wicking or absorbent materials are used to reduce moisture exposure. Materials such as polyester or rubber, having non-neutral charges, set the stage for the development of clinically visible HPV lesion.

Administering the vaccine may be accomplished by intravenous, intramuscular, intradermal, intraperitoneal, subcutaneous, by spray, by aerosol, mucosal, transdermal, by immersion, oral, intraocular, intratracheal, intranasal, pulmonary, or rectal delivery, or other means known to those skilled in the art. In some aspects, the administering comprises oral, epidermal, intradermal intramuscular, topical, intravenous, rectal, or sublingual administration. In some aspects, the vaccine for preventing HPV infection is an anti-HPV 1, 2, 4, 57, 60, 63, 65, 66, or 156, or any combination thereof, vaccine. In some aspects, the anti-HPV vaccine is an HPV vaccine effective against one or more of HPV types 6, 11, 16, and 18. In some aspects, the anti-HPV vaccine is GARDASIL®.

Without being bound by theory, it is believed that HPV can incorporate within the DNA of epithelial cells, including the keratinocytes, and remain dormant. Elimination of HPV containing epithelial cells and/or manifestation of a clinically detectable lesion depend on the interaction of HPV and the keratinocytes, and the immune cells with the keratinocytes. Environmental changes, such as increased moisture, friction, or direct or close contact with non-neutrally electrically charged materials may adversely affect the skin's barrier properties that promote innate protection against HPV. For example, exposure of the affected area to moisture, and in some instances friction, may affect the body's ability to interact defensively and normally with HPV. Such factors may put those susceptible to developing plantar warts, especially but necessarily limited to children and the elderly, at heightened risk, as well as contribute to a patient's response to remedies disclosed herein.

As exposure to moisture may be a contributing factor in the development of a clinically detectable HPV lesion, moisture removal, avoidance, and/or prevention as a means to prevent or reduce plantar warts associated with HPV are contemplated herein. Thus, in some aspects of the present disclosure reducing exposure of the area susceptible to plantar warts to moisture comprises employing at least one mechanical means for removing moisture from the area. In some embodiments, the moisture to which exposure is reduced is sweat moisture. A mechanical means for removing moisture can be, but is not limited to, a towel or other absorbent pad. The mechanical means for removing moisture can comprise a wicking material that removes moisture from the area likely to be affected by plantar warts that either retains said moisture in the fabric or delivers it away from the area. Wicking socks, for example, can remove sweat from the feet to maintain a relatively dry environment compared to wearing non-wicking socks.

Another mechanism for removing moisture includes administering at least one drying agent. For example, placing a desiccant in close proximity to the area of the body likely to be affected by plantar warts can remove problematic moisture. Other drying agents can include, but are not limited to, pharmaceuticals, anti-perspirants, and iontophoresis devices that help prevent, eliminate, alleviate, reduce, or otherwise control excessive perspiration. In some aspects of the invention, exposure to moisture can be prevented, modified, eliminated, alleviated or otherwise controlled by employing a mechanical means for removing moisture and administering a drying agent to remove moisture from the area to maintain the skin, including the stratum corneum, in its natural state.

In some embodiments of the present disclosure, preventing the onset of plantar warts on an area of a patient susceptible to plantar warts comprises administering to the subject a therapeutically effective amount of a vaccine against HPV and instructing the patient to reduce exposure of the area susceptible to plantar warts to moisture, non-neutrally electrically charged materials, or both. Physicians should inform patients that HPV can reside in the epidermal basal layer as a commensal and can even remain dormant within epidermal cells for a person's entire life after infection. Elimination of the HPV-harboring epithelial cells in a timely manner or hindering the development of a clinically detectable plantar wart can be augmented by reducing exposure of potentially affected areas of the skin to moisture and to electrically non-neutral materials. Although individuals' tolerances of moisture exposure and/or electrically non-neutral material varies, the development of a detectable plantar wart in a non-immunocompromised person is a manifestation of excessive exposure to moisture and/or electrically non-neutral material. In some aspects of the present disclosure, non-neutrally electrically charged materials include, but are not limited to, rubber and polyester.

As patients will monitor their health status outside of a clinical setting, it is important that they are informed of the techniques described herein to lessen their likelihood of developing plantar warts. Many subjects may believe that vaccination is completely effective at preventing plantar warts, but individuals exposed to the virus prior to vaccination are still at risk of developing these warts. The delayed onset of clinically detectable HPV lesions may be responsible for an individual falsely assuming they are HPV-free at the time of vaccination. Further to this point, vaccination may also cause complacency, as the vaccinated individual may not be inclined to take any measures to lessen the probability of the onset of warts or HPV-associated symptoms because they assume they are immune to infection. Without taking proper measures to prevent plantar warts, medical intervention may be necessary once the person develops clinically visible verrucae.

The physician or other healthcare provider should provide instructions to the patient that exposure of the feet to moisture should be reduced or eliminated, even if the patient has been previously vaccinated against HPV. Explaining the proper way to dry the area with a mechanical means or to administer a drying agent as described above may accomplish this objective. The physician or other healthcare provider may also prescribe pharmaceuticals or recommend over-the-counter anti-perspiration treatments. The latter may be especially helpful for those with hyperhidrosis.

The physician or other healthcare provider should also explain the impact that friction may have on a latent HPV infection in the areas associated with plantar warts. Without being bound by theory, friction may be a substantial cofactor in the development of clinically detectable plantar warts because it may increase the activation of latent HPV within the basal keratinocytes and/or increase the probability of HPV uptake by the epithelial cells. This activation and/or uptake may result in an increased rate of keratinocyte reproduction. The physician should also instruct the patient to minimize exposure of the area associated with plantar wart to any type of material that may be, or become, non-neutrally electrically charged. Items such as, but not limited to, socks and stockings or clothing manufactured from synthetic fabrics are non-neutrally electrically charged and serve as a predisposing factor to develop clinically visible HPV lesions. As part of the treatment and prevention of plantar warts, such contact with non-neutrally electrically charged materials should be reduced as much as possible, if not eliminated.

Instructions to avoid exposure to moisture and non-neutrally electrically charged clothing would increase the effectiveness of over the counter (OTC) medications for treating plantar warts. For example, OTC plantar wart removal medications containing salicylic acid should contain the following skin and footwear recommendations to improve the effectiveness of treatment and reduce the likelihood of recurrence:
1. Avoid wearing rubber and plastic shoes and substitute with leather (preferably open-toed) with leather or natural fabric insoles.
2. In the case of severe plantar hyperhidrosis, apply aluminum containing antiperspirants or receive iontophoresis therapy.
3. The feet should be kept warm and dry.

Antiperspirants marketed to reduce the likelihood of developing plantar warts or render the treatment of plantar warts more effective should clearly instruct the user to minimize contact with rubber or synthetic material footwear.

Some embodiments of the present disclosure describe methods of treating plantar warts on an affected area of a subject comprising removing at least one plantar wart from the affected area on the subject and administering a drying agent or absorbent composition to the affected area. In some aspects of the embodiment, removing the at least one plantar wart comprises laser removal therapy, liquid nitrogen therapy, or physical excision, and in some aspects removing the at least one plantar wart comprises liquid nitrogen therapy. In some aspects, removing the at least one plantar wart comprises administering a caustic agent including, for example cantharidin or podofilox (e.g., CONDYLOX). In some aspects the caustic agent is cantharidin. In some aspects, removing the at least one plantar wart comprises administering an acid. In some aspects, the acid is salicylic acid, or trichloroacetic acid. In some aspects, removing the at least one plantar wart comprises administering a chemotherapeutic agent. In some aspects, the chemotherapeutic agent comprises 5-fluororuacil, bleomycin, or a combination thereof. In some aspects, removing the plantar wart comprises administering an herbal extract. In some aspects, the herbal extract comprises sinecathechins. In some embodiments, the herbal extract is a caustic herbal agent, for example, ingenol mebutate (e.g., PICATO® gel).

The primary focus of wart treatment has traditionally been infection control measures through destructive modalities, excision, and, more recently, vaccination with synthetic proteins that are found on the capsule of HPVs. Restoration of the natural state of the barrier must be instructed to the patient first, and combined with the currently recommended treatment or preventative modalities to render them much more efficacious. Because asymptomatic individuals may be at risk of developing further warts, some embodiments presently disclosed are directed to methods of preventing recurrence of plantar warts on an area of a subject previously affected with plantar warts comprising administering a drying agent or absorbent composition to the area previously affected with plantar warts. Especially in, but not limited to, instances of recurrence after treatment, a physician or healthcare provider may need to provide additional information to his/her patient to reduce the probability of recurrence. This disclosure contemplates the methods of preventing recurrence as described above as also further comprising instructing the subject to reduce exposure of moisture to the affected area. In addition to all of the above, or in place of instructing the patient to reduce exposure to moisture, some aspects of the embodiment further comprise administering a therapeutic amount of a pharmaceutical composition effective for treating at least one symptom associated with hyperhidrosis to the subject. Administration may be accomplished as described above.

Many individuals will not be vaccinated against HPV. The reasons for not being vaccinated include personal choice, medical reasons such as allergy to egg or other ingredients of the vaccine, or lack of access to the vaccine. Another embodiment contemplated in the present disclosure is a method of preventing plantar warts on an area of the skin in a subject comprising reducing exposure of the area of skin to moisture, which does not require the administration of a vaccine. In some aspects, the moisture to which exposure is to be reduced is sweat moisture. In the absence of a vaccine, reducing the exposure of an area susceptible to plantar warts to moisture may prevent the occurrence of warts. With reduced moisture exposure, keratinocytes are less likely to develop clinically visible verrucae (or warts) than in a high moisture environment. Without being bound by theory, in a reduced moisture content environment, those cells harboring HPV are less likely to develop into a clinically detectable HPV lesion.

An area susceptible to plantar warts may be exposed to moisture from a variety of sources. Of particular importance, is the duration and amount of sweat exposure. An unnatural amount of sweat exposure and the duration of exposure sets the stage for the development of clinically visible verrucae. While moisture reduction is important in decreasing the incidence of plantar warts in most individuals, it is a paramount goal in those individuals with excessive or even just above average perspiration. In some aspects of the present disclosure, reducing exposure of the area susceptible to plantar warts to moisture comprises administering a therapeutically effective amount of a pharmaceutical composition effective for treating at least one symptom associated with hyperhidrosis. Administration of such a pharmaceutical composition is described above.

In some aspects of the present disclosure, a desiccant is used along with other treatment modalities to reduce exposure to moisture. For example, methods of preventing plantar warts may be augmented by further comprising administering a desiccant to clothing or an accessory that occludes the area. In some aspects, the occluding clothing or accessory is a shoe or a sock, but it can be any clothing that can retain moisture. Occluding clothing can include tight fitting clothing or shoes. Examples of tight fitting clothing include, but are not limited to, socks and compression hose. For this reason, reducing exposure to moisture, in some aspects of all of the methods presented herein, comprises wearing non-occlusive clothing. In some aspects, reducing exposure to moisture comprises reducing the wearing of occlusive clothing. In some aspects, reducing exposure to moisture comprises minimizing the wearing of occlusive clothing.

In some aspects, the present disclosure is directed to kits or articles of manufacture. The terms “kit” and “article of manufacture” are used synonymously herein. For use in the methods described herein, kits and articles of manufacture are also described. Such kits include a package or container that is compartmentalized to receive one or more therapeutically effective doses or amounts of a HPV vaccine disclosed herein. In one embodiment, the containers are formed from a variety of materials such as glass or plastic.

The articles of manufacture provided herein can contain packaging materials. Packaging materials for use in packaging pharmaceutical products include, e.g., U.S. Pat. Nos. 5,323,907, 5,052,558 and 5,033,252. Examples of pharmaceutical packaging materials include, but are not limited to, blister packs, bottles, tubes, bags, containers, bottles, and any packaging material suitable for providing one or more therapeutically effective doses or amounts of a vaccine effective against HPV and intended mode of administration and treatment.

A kit typically includes labels listing contents and/or instructions for use, and package inserts with instructions for use. A set of instructions will also typically be included. Thus, the articles of manufacture provided herein can further contain a package insert that contains instructions for treating or preventing plantar warts on an area of a subject susceptible to plantar warts in accordance with the methods described herein, comprising instructions to reduce exposure of the area susceptible to plantar warts to moisture, non-neutrally electrically charged materials, or a combination thereof. The articles of manufacture provided herein can optionally comprise a therapeutically effective amount of at least one desiccant or at least one mechanical means for removing moisture from the area susceptible to plantar warts according to the present disclosure.

In one embodiment, a label is on or associated with the container. In one embodiment, a label is on a container when letters, numbers or other characters forming the label are attached, molded or etched into the container itself; a label is associated with a container when it is present within a package, container, receptacle, or carrier that also holds the container, e.g., as a package insert.

In one embodiment, a label is used to indicate that the contents are to be used for a specific therapeutic application. The label also indicates directions for use of the contents, such as in the methods described herein.

Those skilled in the art will appreciate that numerous changes and modifications can be made to the preferred embodiments of the invention and that such changes and modifications can be made without departing from the spirit of the invention. It is intended that the appended claims cover all such equivalent variations as fall within the true spirit and scope of the invention.

### EMBODIMENTS

The following list of embodiments is intended to complement, rather than displace or supersede, the previous descriptions.

Embodiment 1. A method of preventing plantar warts on an area of a subject susceptible to plantar warts comprising: administering to the subject a therapeutically effective amount of a vaccine against human papillomavirus (HPV); and reducing exposure of the area susceptible to plantar warts to moisture, non-neutrally electrically charged materials, or a combination thereof.

Embodiment 2. The method of embodiment 1, wherein the vaccine against HPV is an HPV 1, 2, 4, 57, 60, 63, 65, 66, or 156 vaccine, or any combination thereof.

Embodiment 3. The method of embodiment 1 or 2, wherein the administering comprises oral, epidermal, intramuscular, topical, intravenous, rectal, or sublingual administration.

Embodiment 4. The method of any one of embodiments 1 to 3, wherein reducing exposure of the area susceptible to plantar warts to moisture comprises employing at least one mechanical means for removing moisture from the area susceptible to plantar warts.

Embodiment 5. The method of any one of embodiments 1 to 3, wherein reducing exposure of the area susceptible to plantar warts to moisture comprises administering at least one drying agent.

Embodiment 6. The method of any one of embodiments 1 to 3, wherein reducing exposure of the area susceptible to plantar warts to moisture comprises employing at least one mechanical means and administering at least one drying agent to remove moisture from the area susceptible to plantar warts.

Embodiment 7. A method of preventing the onset of plantar warts on an area of a patient susceptible to plantar warts comprising: administering to the patient a therapeutically effective amount of a vaccine against human papilloma virus (HPV); and instructing the patient to reduce exposure of the area susceptible to plantar warts to moisture, non-neutrally electrically charged materials, or both.

Embodiment 8. The method of any one of embodiments 1 to 7, wherein the vaccine is effective against one or more of HPV types 6, 11, 16, and/or 18, e.g., GARDASIL®.

Embodiment 9. The method of embodiment 7 or 8, wherein the administering comprises oral, epidermal, intramuscular, topical, intravenous, rectal, or sublingual administration.

Embodiment 10. The method of any one of embodiments 7 to 9, wherein instructing the patient to reduce exposure of the area susceptible to plantar warts to moisture comprises instructing the patient to employ at least one mechanical means for removing moisture from the region.

Embodiment 11. The method of any one of embodiments 7 to 10, wherein instructing the patient to reduce exposure of the area susceptible to plantar warts to moisture comprises instructing the patient to administer at least one drying agent.

Embodiment 12. The method of any one of embodiments 7 to 11, wherein instructing the patient to reduce exposure of the area susceptible to plantar warts to moisture comprises instructing the patient to employ at least one mechanical means and administering at least one drying agent to remove moisture from the area susceptible to plantar warts.

Embodiment 13. A method of preventing recurrence of plantar warts on an area of a subject previously affected with plantar warts comprising administering a drying agent or absorbent composition to the area previously affected with plantar warts.

Embodiment 14. A method of treating plantar warts on an affected area of a subject comprising: removing at least one plantar wart from the affected area on the subject; and administering a drying agent or absorbent composition to the affected area.

Embodiment 15. The method of embodiment 14, wherein removing the at least one plantar wart comprises administering laser removal therapy, liquid nitrogen therapy, at least one caustic agent, at least one acid, at least one herbal extract, at least one chemotherapeutic agent, or physical excision, or a combination thereof.

Embodiment 16. The method of embodiment 15, wherein removing the at least one plantar wart comprises administering liquid nitrogen therapy.

Embodiment 17. The method of embodiment 14, wherein removing the at least one plantar wart comprises administering a caustic agent.

Embodiment 18. The method of embodiment 17, wherein the caustic agent is cantharidin. In other embodiments, the caustic agent is podofilox, e.g., CONDYLOX®. In other embodiments, the caustic agent comprises one or both of cantharidin or podofilox.

Embodiment 19. The method of embodiment 14, wherein the removing the at least one plantar wart comprises administering at least one acid.

Embodiment 20. The method of embodiment 19, wherein the at least one acid is salicylic acid, trichloroacetic acid, or a combination thereof.

Embodiment 21. The method of embodiment 14, wherein removing the at least one plantar wart comprises administering at least one herbal extract.

Embodiment 22. The method of embodiment 21, wherein the at least one herbal extract comprises sinecathecins. In other embodiments, the at least one herbal extract comprises ingenole mebutate, e.g., PICATO® gel. In other embodiments, the at least one herbal extract comprises both sinecathecins and ingenole mebutate.

Embodiment 23. The method of embodiment 14, wherein removing the at least one plantar wart comprises administering a chemotherapeutic agent.

Embodiment 24. The method of embodiment 19, wherein the chemotherapeutic agent is 5-fluorouracil or bleomycin.

Embodiment 25. The method of any one of embodiments 14 to 22, further comprising instructing the subject to reduce exposure of moisture to the affected area.

Embodiment 26. The method of any one of embodiments 14 to 23 further comprising administering to the subject a therapeutic amount of a pharmaceutical composition effective for relieving at least one symptom associated with hyperhidrosis.

Embodiment 27. The method of embodiment 24, wherein administering a pharmaceutical composition effective for relieving symptoms associated with hyperhidrosis comprises oral, epidermal, intramuscular, topical, intravenous, rectal, or sublingual administration.

Embodiment 28. A method of preventing plantar warts on an area of a subject susceptible to plantar warts comprising reducing exposure of the area susceptible to plantar warts to moisture.

Embodiment 29. The method of embodiment 28, wherein the moisture is sweat moisture.

Embodiment 30. The method of embodiment 28, wherein reducing exposure of the area susceptible to plantar warts to moisture comprises administering a therapeutically effective amount of a pharmaceutical composition effective for relieving at least one symptom of hyperhidrosis.

Embodiment 31. The method of embodiment 30, administering a pharmaceutical composition effective for relieving at least one symptom associated with hyperhidrosis comprises oral, epidermal, intramuscular, topical, intravenous, rectal, or sublingual administration.

Embodiment 32. The method of any one of embodiments 28 to 31, further comprising administering a desiccant to any clothing or accessory that occludes the area susceptible to plantar warts.

Embodiment 33. The method of embodiment 32, wherein the occluding clothing or accessory is a shoe, a sock, or compression hose.

Embodiment 34. The method of any one of embodiments 13 to 33 further comprising administering a therapeutically effective amount of a vaccine effective against human papilloma virus.

Embodiment 35. The method of embodiment 34, wherein the vaccine effect against one or more of human papillomavirus type 6, 11, 16, and 18, e.g., GARDASIL®.

Embodiment 36. The method of any one of embodiments 1 to 12 or 23 to 35, wherein reducing moisture comprises minimizing wearing non-electrically-neutral shoes or socks, such as rubber made sportswear, to a few hours per day or even less in the case of plantar hyperhidrosis.

Embodiment 37 A kit comprising: a) a therapeutically effective amount of a vaccine effective against human papilloma virus; b) a package insert that contains instructions for treating or preventing plantar warts on an area of a subject susceptible to plantar warts comprising instructions to reduce exposure of the area susceptible to plantar warts to moisture, non-neutrally electrically charged materials, or a combination thereof; and c) optionally, a therapeutically effective amount of at least one desiccant or at least one mechanical means for removing moisture from the area susceptible to plantar warts.

1. A method of preventing plantar warts on an area of a subject susceptible to plantar warts comprising:
administering to the subject a therapeutically effective amount of a vaccine against a human papilloma virus (HPV); and one or both of the following:
i) reducing exposure of the area susceptible to plantar warts to moisture, non-neutrally electrically charged materials, Of a combination thereof; and
ii) instructing the subject to reduce exposure of the area susceptible to plantar warts to moisture, non-neutrally electrically charged materials.

2. The method of claim 1, wherein the vaccine against HPV is an HPV 1,2,4, 57, 60, 63, 65, 66, or 156 vaccine, or any combination thereof.

3. The method of claim 1, wherein the administering comprises oral epidermal, intramuscular, topical, intravenous, rectal, or sublingual administration.

4. The method of any of claim 1, wherein reducing exposure of the area susceptible to plantar warts to moisture comprises employing at least on mechanical means for removing moisture from the area susceptible to plantar warts.

5. The method of any one of claim 1, wherein reducing exposure of the area susceptible to plantar warts to moisture comprises administering at least one drying agent.

6. The method of any of claim 1, wherein reducing exposure of the area susceptible to plantar warts to moisture comprises employing at least one mechanical means and administering at least one drying agent to remove moisture from the area susceptible to plantar warts.

7. (canceled)

8. (canceled)

9. (canceled)

10. (canceled)

11. (canceled)

12. (canceled)

13. (canceled)

14. A method of treating plantar warts on an affected area of a subject comprising:
a. removing at least one plantar wart from the affected area on the subject; and
b. administering a drying agent or absorbent composition to the affected area.

15. The method of claim 14, wherein removing the at least one plantar wart comprises administering laser removal therapy, liquid nitrogen therapy, at least one caustic agent, at least one acid, at least one herbal extract, at least one chemotherapeutic agent, or physical excision, or a combination thereof.

16. The method of claim 15, wherein removing the at least one plantar wart comprises administering liquid nitrogen therapy.

17. The method of claim 14, wherein removing the at least one plantar wart comprises administering a caustic agent.

18. The method of claim 17, wherein the caustic agent is cantharidin.

19. The method of claim 14, wherein the removing the at least one plantar wart comprises administering at least one acid.

20. The method of claim 19, wherein the at least one acid is salicylic acid, trichloroacetic acid, or a combination thereof.

21. The method of claim 14, wherein removing the at least one plantar wart comprises administering at least one herbal extract.

22. The method of claim 21, wherein the at least one herbal extract comprises sinecathecins or ingenol mebutate.

23. The method of claim 14, wherein removing the at least one plantar wart comprises administering a chemotherapeutic agent.

24. The method of claim 19, wherein the chemotherapeutic agent is 5-fluorouracil or bleomycin.

25. The method of any one of claims 14, further comprising instructing the subject to reduce exposure of moisture to the affected area.

26. The method of any one of claims 14, further comprising administering to the subject a therapeutic amount of a pharmaceutical composition effective for relieving at least one symptom associated with hyperhidrosis.

27. (canceled)

28. (canceled)

29. (canceled)

30. (canceled)

31. (canceled)

32. (canceled)

33. (canceled)

34. (canceled)

35. (canceled)

36. (canceled)

37. A kit comprising:
a. a therapeutically effective amount of a vaccine effective against human papilloma virus;
b. a package insert that contains instructions for treating or preventing plantar warts on an area of a subject susceptible to plantar warts comprising instructions to reduce exposure of the area susceptible to plantar warts to moisture, non-neutrally electrically charged materials, or a combination thereof; and
c. optionally, a therapeutically effective amount of at least one desiccant or at least one mechanical means for removing moisture from the area susceptible to plantar warts.
